(19) United States
(12) Patent Application Publication
(10) Pub. No.: US 2014/0303300 A1
(43) Pub. Date: Oct. 9, 2014

(54) METHOD FOR CONTINUOUS NUCLEOPHILIC ADDITION TO ACTIVATED CARBON-CARBON MULTIPLE BONDS

(71) Applicants: Jörg Issberner, Willich-Neersen (DE); René Nagelsdiek, Hamminkeln (DE); Frank Tlauka, Oberhausen (DE); Bemd Göbelt, Wesel (DE); Tom Beckmann, Hamminkeln (DE)

(72) Inventors: Jörg Issberner, Willich-Neersen (DE); René Nagelsdiek, Hamminkeln (DE); Frank Tlauka, Oberhausen (DE); Bemd Göbelt, Wesel (DE); Tom Beckmann, Hamminkeln (DE)

(73) Assignee: BYK-Chemie GmbH, Wesel (DE)

(21) Appl. No.: 14/240,300

(22) PCT Filed: Aug. 23, 2012
(86) PCT No.: PCT/EP2012/066426

§ 371 Date: May 13, 2014

(30) Foreign Application Priority Data
Aug. 23, 2011 (EP) 11178446.8

(51) Int. Cl. C07F 7/10; C07B 43/04; C07C 227/06

(52) U.S. Cl. CPC C07F 7/10; C07C 227/06; C07B 43/04
USPC 524/188; 556/413; 560/252

(57) ABSTRACT

The invention relates to a method for continuous production of reaction products by addition reactions based on a Michael reaction, wherein at least one compound (B) having at least one nucleophilic functional group is added to at least one compound (A) having at least one activated alkene or activated alkyne carbon-carbon multiple bond, wherein the reaction takes place in a reaction mixing pump.

The invention relates to a method for the continuous addition of nucleophiles onto compounds with activated carbon-carbon multiple bonds. Reactions of this type are also referred to in the literature as Michael additions or Michael reactions.

In the narrower sense, a Michael reaction is a reaction between a carbanion as nucleophilic reactant and an activated carbon-carbon double bond, i.e. a carbon-carbon double bond substituted with at least one electron-withdrawing group as electrophilic acceptor with new linkage of a carbon-carbon single bond. The present invention is based on a more broadly defined definition of the Michael reaction or Michael addition. Here, the nucleophilic reactant can, for example, also be an amine or thiol instead of a carbanion, resulting in the new linkage of a nitrogen-carbon or sulfur-carbon bond. In the context of this invention, the electrophilic acceptor is not limited to an activated carbon-carbon double bond, but may also be an activated carbon-carbon triple bond, in which case the addition product then in turn contains a carbon-carbon double bond. In each case, the covalent bond linkage takes place between a donor (nucleophile, “Michael donor”) and an activated, electrophilic alkene or alkyne (“Michael acceptor”). The definitions of the narrower and broader understanding of a Michael reaction are widespread and exemplified for example also in Volume 2 of the Lehrbuch für Lacke and Beschichtungen [Textbook of paints and coatings] from the author H. Kittel (S. Hirzel Verlag Stuttgart/Leipzig 1998, 2^{nd} edition, chapter 2.2.3.3 “Reactive systems based on the Michael reaction”, pages 328 to 334), in Eur. J. Org. Chem. 2010, 2009-2006 or in Prog. Polymer Sci. 2006, 31, 487-531.

Since a single bond is formed in the reaction of a carbon-carbon double bond with a Michael donor from the double bond, and a double bond is formed in the reaction of a carbon-carbon triple bond with a Michael donor from the triple bond, the addition reaction in question is highly exothermic, which brings with it the problem of heat dissipation during technical implementation. At the same time, the exothermy also constitutes a safety risk, particularly if one considers that alkenically or alkynically unsaturated compounds are present in high concentration as unreacted reactants, which brings with it the risk of a sudden reaction in the course of a radical chain reaction under certain circumstances. In addition to the safety risk, such secondary reactions, according to experience, also adversely affect the quality of the resulting product (e.g. in that molecules with a considerably higher molecular weight are formed).

For the addition products in question to be able to be used, however, it is particularly important to obtain qualitatively high-value products which are largely free from undesired byproducts. Furthermore, it is desirable to dissipate the amount of heat liberated during the addition reaction as effectively as possible in a safe method, so that an undesired polymerization (“autopolymerization”) of the alkenically or alkynically unsaturated component, which not only brings with it a change in product properties, but in particular entails a high risk for plant safety, can be excluded. Such heat dissipation can take place by classic methods e.g. by working in dilute solution, as a result of which corresponding amounts of a solvent are entrained. However, since these solvents are undesired for many applications (many solvents are classed as hazardous materials and in many applications, volatile organic compounds (VOC) are not acceptable), they have to be removed again in this case in their entirety by means of complex methods. The removal is associated with time expenditure and operating costs, and moreover the amounts of removed solvent produced in the process in many cases cannot be used further, but have to be disposed of, and are thus lost resources. Moreover, the conditions required for removing the solvent (e.g. relatively high temperatures) can adversely affect the quality of the actual product.

DE 4220239 A1 from 1993 describes a mixing device with which homogeneous and stable mixtures can be obtained from two or more preferably reactive, liquid components. An example of a liquid component given is solvents in which further organic products can be dissolved. DE 4220239 A1 focuses here on the mixing operation. However, a known and desired reaction implementation in the mixing device itself, especially between Michael donors and Michael acceptors in the sense of this invention, is not described. The suitability of this mixing device as the reactor itself, in which a significant proportion of the reactive components are reacted, is likewise not disclosed. There is just as little discussion of the option of external thermal regulation by means of heating or cooling units, which option is required for a targeted reaction implementation.

By contrast, WO 2010/133292 A1 discloses a method for the continuous production of epoxy amine compounds. To carry out the method, according to WO 2010/133292 A1, any desired continuously operable reactors can be used, with a mixing pump, in which some of the reaction between epoxide component and amine component can also proceed, only being used in a specific embodiment. A transferability of this specific processing technology to the Michael addition of the present invention with the problems typical of a Michael addition of a possible autopolymerization of the alkenically or alkynically unsaturated compounds used is neither disclosed nor in any way suggested in WO 2010/133292 A1. For the average skilled person experienced in the field of Michael addition, there was thus no reason to consider a processing measure which was described for a reaction between such components which per se does not acknowledge the problems of an autopolymerization.

It was an object of the present invention to provide a method for the continuous production of Michael addition products which does not have the disadvantages of the prior art. In particular, local overheatings should be avoided in order to ensure the required extremely high product qualities and to avoid continuing reactions such as, for example, a polymerization risk. Nevertheless, a high space-time yield should be achieved here.

Mixing techniques which render it necessary to implement two or more active mixing apparatuses in a plurality of reactor segments or reactors combined with one another, should be avoided on account of maintenance and economic feasibility reasons in particular. Nevertheless, the method should ensure that it is possible to use not only mutually soluble, low molecular weight or low viscosity starting materials. Instead, products should also be obtainable in high quality and good yield if they are obtained from high viscosity starting materials or starting materials which have poor mutual solubility without having to use larger amounts of solvent.

Moreover, the method should permit a rapid and exact adjustment of the reaction conditions in order, in the event of a shortened reaction time, to nevertheless obtain improved or at least substantially identical selectivities and yields as is possible with the methods in the prior art. As a result of the shortened reaction times of the method to be provided, the aim was also to open up the possibility of carrying out the reactions at considerably higher temperatures than is possible for discontinuous methods on account of the long reactor residence times. The aim was also to provide the possibility of reacting gaseous starting materials with liquid starting materials. In particular, the method should also permit a safer production of the target products, in which case this production should be possible in the absence of solvents.

The objects and problems mentioned above and also discussed in the description below were able to be solved by providing a method for the continuous production of reaction products by addition reaction, where at least one compound (B), which has at least one nucleophilic functional group, is added onto at least one compound (A), which has at least one activated alkenic or activated alkynic carbon-carbon multiple bond, where the reaction takes place in a reaction mixing pump.

This type of addition reaction is an addition reaction based on the Michael reaction. Addition reactions of the present invention are to be distinguished from hydrosilylation reactions, which are already therefore not in accordance with the invention since during a hydrosilylation the functional group Si—H is unable to act as a donor for a carbon-carbon multiple bond on account of the electropositivity of the silicon atom. In the addition reactions based on the Michael reaction according to the present invention, therefore, carbon-carbon, carbon-nitrogen, carbon-sulfur or carbon-phosphorus single bonds are therefore very particularly preferably newly formed.

Reaction mixing pumps for the purposes of this invention are preferably of the peripheral wheel pump type and are equipped with
(a) a rotationally symmetrical mixing chamber composed of a circumferential wall and two faces, which have annular channels fluidically joined to one another,
(b) at least one inlet opening to the mixing chamber, via which the compound(s) (A) are introduced,
(c) at least one inlet opening to the mixing chamber, via which compound(s) (B) are introduced,
(d) a magnet-coupling-driven mixing rotor in the mixing chamber, which has edge breaks symmetrically arranged on the face and which form pressure cells with the annular channels on the faces of the mixing chamber, and where the pressure cells are joined together via connecting bores in the mixing rotor,
(e) an outlet opening of the mixing chamber, via which the reaction mixture and/or the product are discharged from the reaction mixing pump, and
(f) a thermally regulatable circuit thermally regulatable by means of an external heating or cooling unit.

A rotation mixing pump suitable for the method according to the invention is described for example in DE-A-42 20 239 that has already been mentioned above and which is incorporated into this application by citation. The pump head, however, is additionally, for the purposes of the present invention, designed to be thermally regulatable via a thermally regulatable circuit by means of an external heating or cooling unit. The periphery additionally consists at least of an optionally heatable dosing device for each starting material and a downstream optionally heatable line for the reaction mixture.

Further different configurations of reaction mixing pumps that can be used according to the invention are commercially available for example from K-ENGINEERING (Westoverledingen, Germany) under the name “HMR”. These devices combine the properties of a peripheral wheel pump, a mixer for particularly effective thorough mixing and a reactor. As a result of this, the method according to the invention requires a very low expenditure on apparatuses. The mixing chambers of the reaction mixing pump(s) that can be used according to the invention comprise a bearing support and a cylindrical insert element with a circumferential wall covering the mixing rotor. The circumferential wall of the reaction mixing pump has at least one inlet opening for each of the compounds (A) and compounds (B), and an outlet opening for the reaction mixture.

It has proven to be particularly favorable to design the entry openings of the reaction pumps tapered nozzle-like in the direction of the reaction mixing chamber because a type of suction effect arises as a result.

The rotational frequency of the rotor, which is expediently controlled via an external frequency converter, is usually 50 to 50 000 revolutions per minute while carrying out the method according to the invention.

The reaction volume in the reaction mixing pump is usually 1 to 1000 cm³, preferably 1 to 100 cm³ and particularly preferably 5 to 100 cm³.

It goes without saying that the parts of the reaction mixing pump which come into contact with the starting materials, the reaction mixture and the reaction product must be manufactured from, or coated with, a material which is inert towards the constituents of the reaction mixture. Such materials are, for example, metals or metal alloys, such as Hastelloy, titanium or nickel, plastics, such as polyethylene (PE), polypropylene (PP), polyvinylidene fluoride (PVDF) or in particular polytetrafluoroethylene (PTFE), or oxide ceramic.

Reaction pumps are generally designed in technical terms to correspond to the desired pressure ratios in the mixing chamber.

If larger amount of the target product are required, then it is also possible to operate a plurality of reaction mixing pumps connected in parallel as a reaction mixing pump unit (in the sense of so-called “numbering-up”). For this purpose, the reaction mixing pump is arranged in a device which contains further, in each case independently of one another continuously operated reaction mixing pumps in which the compound(s) (A) are reacted with the compound(s) (B), where the reaction mixing pumps can be operated in parallel at the same time and independently of one another. Such a parallel operation ensures not only the generation of high production amounts, but also a high flexibility since it is possible to replace a defective reaction mixing pump operating in this way by another quickly and with relatively little effort. Compared to batch methods, greater reactor safety is also ensured since in the case of technical problems, the risk of relatively large amounts of starting material, reaction mixture and product escaping is avoided.

The reaction mixing pumps used in the method according to the invention can moreover be equipped with further connection options for heating, cooling and rinsing circuits.

Reaction mixing pumps described above increase the rate of the mass transfer and heat transfer processes, where additionally initial and edge conditions of the reaction can be adjusted exactly. The residence times can be adjusted particularly precisely, where the highly exothermic method according to the invention can be operated approximately isothermally and preferably simultaneously at a low temperature.

Typical operating parameters of the reaction mixing pumps that can be used in the method according to the invention are their throughput of preferably 100 ml/h to 1000 l/h, particularly preferably 100 ml/h to 10 l/h, the temperature in the reaction mixing pump of preferably −50 to 300° C., particularly preferably 50 to 250° C., the pressure in the reaction mixing pump of preferably 0 to 20 bar, particularly preferably 0 to 10 bar, the revolutionary speed of the rotor of preferably 50 to 50000 revolutions per minute, particularly preferably 500 to 10000 revolutions per minute, the residence time in the reaction mixing pump of preferably 0.1 second to 30 min, preferably up to 10 min and particularly preferably up to 1 min.

In a particular embodiment, further reactor systems operated in a continuous manner are connected downstream of the reactor, which reactor systems can effect a post-addition of the compound(s) (A) and/or of the compound(s) (B) and/or a post-thermal regulation for the purposes of completing the reaction. The post-reaction in the downstream reactor systems ensures in some cases only the attainment of the desired conversion, which, based on the nucleophilic donor functions that can be converted in total, is typically preferably at least 30%, particularly preferably at least 70% and very particularly preferably 95% and higher.

In the simplest and most preferred case, a post-reactor is designed in the form of a tube or pipe, in each case made of material that is inert towards the starting materials, the reaction mixture and the products. If simple post-reactors of this type are used, it is usually not necessary to use further mixing devices in the post-reactor on account of the already excellent thorough mixing in the reaction mixing pump. Typical post-reaction times are from 0 to 100 min, preferably 0 to 50 min and particularly preferably 5 to 30 min.

In a particular embodiment, the downstream reactors used can also be reaction mixing pumps as used in the method according to the invention. The product stream from the reaction mixing pump used in the method according to the invention is then one of the entry streams or starting material streams for the downstream reactor. Thus, for example reactive functional groups in the product of the method according to the invention can be reacted with other compounds in the post-reactor.

The reaction products of donor compound(s) (B) and acceptor compound(s) (A) are in the form of addition compounds. Preferably, in the reaction mixing pump 10 to 100 mol % of the alkenically or alkynically unsaturated functions introduced as a result of introducing the electrophilic acceptor compound(s) (A) into the reactor are reacted in the reactor. In some embodiments of the method, it may be preferred that for example only 10 to 50 mol % or 20 to 50 mol % of the alkenically or alkynically unsaturated functions introduced as a result of introducing the acceptor compound(s) (A) into the reactor are reacted in the reactor. This is the case particularly when post-reactors are used as described above. In some cases, it is also possible in this way to minimize the residence time in the reaction mixing pump when namely, as a result of the partial formation of the product, a dissolution or emulsification of the starting materials in the product (in addition to the active mixing by the pump) contributes to the homogenization of the reactants. In all cases, however, a minimum proportion of the compounds with alkenically or alkynically unsaturated groups introduced continuously into the reactor still react primarily (or optionally also exclusively) with the compound(s) (B) in the reaction mixing pump itself. In this regard, the process according to the invention ensures the reaction mixture has an adequately long residence time in the reactor. To determine the degree of conversion, all customary optical analytical methods are suitable, such as, for example, Raman spectroscopy, UV spectroscopy, IR spectroscopy or NIR spectroscopy, and also NMR spectroscopy, in particular coupled with chromatographic methods such as gel permeation chromatography or HPLC. Of particular suitability is UV- and also NIR-spectroscopic observation of the disappearance of the band of the carbon-carbon multiple bond.

It is essential that the method according to the invention is relatively easy to handle, as a result of which the underlying highly exothermic reaction can be well controlled. Particularly in continuous operation, the method according to the invention ensures high economic feasibility and operational safety. At the same time, the undesired formation of byproducts as a result of free radical polymerization is suppressed.

Via the compound(s) (B), property-modifying radicals are inserted into the alkenically or alkynically unsaturated compounds (A) which co-determine the intended use of the target product.

As compound(s) (A), compounds are used which have activated carbon-carbon multiple bonds. Suitable carbon-carbon multiple bonds are C═C double bonds and C≡C triple bonds, preference being given to using those compounds which have one or more C═C double bonds. The carbon-carbon multiple bonds can be present in the compounds (A) in the terminal position or in another position in the compound, preference being given to terminal carbon-carbon multiple bonds. Particular preference is given to compounds (A) which contain terminal C═C double bonds.

No carbon-carbon multiple bonds for the purposes of this invention are aromatic carbon-carbon bonds. Thus, for example in benzyl acrylate, only one carbon-carbon multiple bond in the sense of the invention is present, namely that of the acrylate radical in benzyl acrylate.

The compounds (A) can preferably be represented by the general structural formulae (Ia) and (Ib):

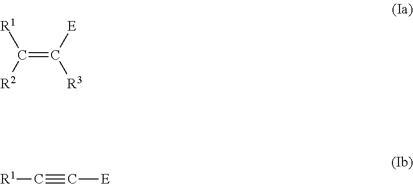

in which
E is an electron-withdrawing substituent, and R¹, R² and R³, independently of one another, are H, E, an aliphatic, aromatic or aliphatic-aromatic radical, and where R¹ and E can be joined together by ring closure. The electron-withdrawing substituent E activates, by virtue of its presence, the adjacent C═C double bond or C≡C triple bond in the sense of the present invention.

If certain radicals—such as for example the aforementioned radicals R¹, R² and R³ or the radicals R, R⁴, R′ and R^{z} mentioned below—are referred to in the present invention in general terms as being aliphatic, aromatic or aliphatic-aromatic, then these may in general be monomeric, oligomeric or polymeric radicals. Unless expressly mentioned otherwise, all radicals can be substituted or unsubstituted and they may also be oligomeric or polymeric radicals.

Preferred electron-withdrawing substituents E are, for example, COR, COOR, CONHR, CONR₂, CN, PO(OR)₂, pyridyl, SOR, SOOR, F or NO₂, where the radicals R, independently of one another, are H or an aliphatic, aromatic or aliphatic-aromatic radical. Most preferably, the electron-withdrawing substituent E contains a multiple bond which is in conjunction with the activated carbon-carbon multiple bond, meaning that the latter is an activated conjugated carbon-carbon multiple bond. Preferably, R is an aliphatic radical, particularly preferably a branched or unbranched alkyl radical having 1 to 12 carbon atoms. In another preferred embodiment, R is an aromatic radical having 6 to 10 carbon atoms, such as, for example, a phenyl radical. In a further preferred embodiment, R is an aliphatic-aromatic radical, such as, for example, a benzyl radical. In a further particularly preferred embodiment, the radical R is a polyether radical, a polyester radical or a polyether-polyester radical. The radicals R themselves can be substituted or unsubstituted. Suitable substituents are in particular functional groups such as, for example, carboxylic acid groups, carboxylic acid ester groups, carboxylic acid amide groups and hydroxyl groups.

R¹, R² and R³ in the general formulae (la) and (Ib), independently of one another, are H, E, an aliphatic, aromatic or aliphatic-aromatic radical, according to the above definitions for R.

Preference is given to the alkenically unsaturated compounds of the general formula (Ia). Among these, particular preference is given to those for which at least two of the radicals R¹, R² and R³ are hydrogen.

If all three radicals R¹, R² and R³ in the general formula (Ia) are hydrogen, then these are vinylically unsaturated compounds (Ia). According to the invention, vinylically unsaturated compounds are most particularly preferred. Typical and preferred representatives among these are acrylic acid, acrylic acid esters (=acrylates), acrylic acid amides (=acrylamides), acrylonitrile, vinylsulfones, vinylphosphonates, vinyl ketones, vinyl aldehydes, vinylpyridines and nitroethylene.

If the radicals R¹ and R² in the general formula (Ia) are hydrogen, R³ is methyl and E is a radical COOR, CONHR, CONR₂, CN (each as defined above), then one speaks of methyacrylic acid (R═H) and its esters, amides and nitriles. Typical and preferred representatives among these are methacrylic acid, methacrylic acid esters (=methacrylates), methacrylic acid amides (=methacrylamides) and methacrylonitrile. If a hydrogen atom is substituted on the methyl radical R³, for example by a further electron-withdrawing group E, then in the case E=COOR, CONHR or CONR₂, one arrives at the likewise usable itaconic acid (R═H), its esters or amides.

In a further preferred embodiment, in which likewise precisely two of the three radicals R¹, R² and R³ are hydrogen, the third radical that is not hydrogen is a further group E. A typical example thereof are the cyanoacrylates (R¹═R²═H, R³═CN and E=COOR) or maleic acid, its esters and amides (R²═R³═H, R¹═COOR or CONHR or CONR₂, E=COOR or CONHR or CONR₂). This embodiment also includes the preferred option of the joining of radicals R¹ and E by ring closure formation. Thus, the radicals R¹ and E can for example form the common radical CO—NR—CO, which in the case of (R²═R³═H) leads to the most particularly preferred maleimides (see following formula (Ia′)).

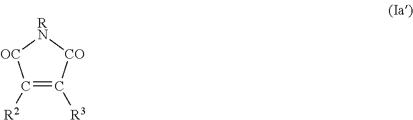

Among the compounds of the general formula (Ib), preference is given to those in which E is a keto group or ester group. R¹ in compounds of the general formula (Ib) is likewise preferably E.

Typical representatives of the compounds (Ib) are beta-ketoacetylenes and acetylene esters (mono- and diesters).

Preferably, the compounds (A) are those selected from the group comprising (meth)acrylate, (meth)acrylamide, acrylonitrile, maleic acid, its esters, amides and imides, itaconic acid, its esters and amides, cyanoacrylates, vinylsulfones, vinylphosphonates, vinyl ketones, nitroethylenes, α,β-unsaturated aldehydes, vinylpyridines, β-ketoacetylenes and acetylene esters. As usual, the notation “(meth)acrylic” includes both “acrylic” and “methacrylic”.

Particular preference is given to the compounds (A) selected from the group comprising (meth)acrylates, (meth)acrylamides, maleic acid esters and maleimides and vinylphosphonates.

In the compounds (A), very particular preference is given to those selected from the group comprising acrylates, acrylamides and maleic acid esters.

The compounds (A) here are preferably those which have the carbon-carbon multiple bonds in terminal position.

Particularly preferably, the compounds (A) contain two or more radicals CR¹R²═CR³COO, where R¹, R² and R³, independently of one another, are as defined above. These radicals CR¹R²═CR³COO are then introduced into the compounds (A) of the general formula (Ia) via the radical E, where E is COOR and the radical R contains one or more of the groups CR¹R²═CR³COO. Examples of such compounds (A) with two radicals CR¹R²═CR³COO are hexanediol diacrylate (HDDA), dipropylene glycol diacrylate (DPGDA) and tripropylene glycol diacrylate (TPGDA). Those with three or four radicals CR²—CR³COO are, for example, trimethylolpropane triacrylate (TMPTA), pentaerythritol triacrylate (PETA), pentaerythritol tetraacrylate and ditrimethylolpropane tetraacrylate (DTMPTTA). In the aforementioned cases, when E is COOR, the radicals R are short-chain monomeric aliphatic radicals preferably with a number-average molecular weight of less than 1500 g/mol, particularly preferably less than 1000 g/mol, very particularly preferably less than 500 g/mol, which carry one, two or three CR¹R²═CR³COO groups. The aforementioned compounds (A) are often also referred to as so-called reactive thinners (see for example Rompp Lexikon Lacke & Druckfarben [paints and printing inks], Georg Thieme Verlag 1998, page 491, keyword “Reaktivverdünner [reactive thinners]”).

However, it is also possible that when E is COOR, the radical R is an oligomeric or polymeric group, for example a polyether or polyester radical which carries further CR¹R²═CR³COO groups. In such a case, they are preferably polyether acrylates or polyester acrylates. Examples of polyether acrylates are polyethylene glycol monoacrylates and polyethylene glycol diacrylates, polypropylene glycol monoacrylates and polypropylene glycol diacrylates, mixed polyethylene glycol/propylene glycol mono- and diacrylates, neopentyl glycol diacrylate, diacrylates of alkoxylated neopentyl glycol, glycerol triacrylate, triacrylates of alkoxylated glycerol, trimethylolpropane triacrylate, triacrylates of alkoxylated trimethylolpropane, bisphenol A diacrylate or diacrylates of alkoxylated bisphenol A. However, so-called epoxy acrylates or urethane acrylates can also be used. Furthermore, it is possible that when E is COOR, the radical R is a radical containing a polysiloxanes chain, preferably a polydimethylsiloxane chain.

In a particularly preferred embodiment, monomeric, oligomeric or polymeric compounds (A) contain at least two (meth)acrylate groups, even more preferably 2 to 8 or most particularly preferably 2 to 4 (meth)acrylate groups.

Compounds (B) are nucleophilic donor compounds.

The compounds (B) can preferably be depicted by the general structural formula (II):

R⁴-D-H (II)

in which R⁴ is an aliphatic, aromatic or aliphatic-aromatic radical and D is CR′E, NR′, PR′ or S, where R′ is hydrogen or an aliphatic, aromatic or aliphatic-aromatic radical and E is an electron-withdrawing substituent. The electron-withdrawing substituent is as defined in the general structural formulae (Ia) and (Ib). The electron-withdrawing substituent E in the radical CR′EH permits the deprotonation of the hydrogen radical bonded to the carbon atom, which is also referred to in the chemical specialist literature as CH-acidic hydrogen, with the formation of a so-called carbanion. As already mentioned at the start, carbanions are Michael donors in the narrower sense. If the electron-withdrawing substituent E is COR, COOR, CONHR or CONR₂, where the radicals R, independently of one another, are H or an aliphatic, aromatic or aliphatic-aromatic radical, then the carbanion structure can rearrange, with inclusion of the group CO, into the tautomeric enolate structure. Particularly preferably, D is NR′, PR′ or S, very particularly preferably NR′ or S. The radical R⁴ can itself in turn contain D-H groups. If in the general formula (II) D-H is an amino group and the radical R⁴ contains further D-H groups in the form of amino groups, then the compounds of the general formula (II) are polyamines. Typical polyamines which fall under the formula (II) are for example diethylenetriamine and triethylenetetramine. It is also possible that they are polyamines with amino groups of differing reactivity, for example mixed primary/secondary amines such as N-methylaminopropylamine (in which the primary amino group is generally more rapidly accessible to the addition) or mixed primary/tertiary amines such as N,N-dimethylaminopropylamine, in which an amino group is available for the addition and the other (tertiary) amino group can no longer add onto an activated C—C multiple bond.

It is furthermore possible and in many cases preferred that the radical R⁴ contains different functional groups, such as, for example, silyl groups, in particular hydrolyzable silyl groups. A preferred radical R⁴ is for example a radical R⁵—Si(R⁶)ₙ(R⁷)₃₋ₙ in which R⁵ is an alkylene radical having 1 to 6, preferably 1 to 3, carbon atoms, R⁶ is a radical that can be cleaved off by hydrolysis, for example a halogen radical, an alkoxy radical having 1 to 4 carbon atoms or an acetyl radical, R⁷ is an alkyl radical having 1 to 6 carbon atoms and n=1 to 3. In particular, hydrolyzable silyl groups in the radical R⁴ are preferred if the radical D is a group NR′ or S.

A further functional group which the radical R⁴ can have are hydroxy groups. This is particularly preferred when the radical D is a group NR′, meaning that the Michael addition reaction is the addition of a so-called amino alcohol onto an activated carbon-carbon multiple bond.

In particular, the nucleophilic donor compounds (B) include primary and secondary amines, thiols, phosphines, and carbanion-forming compounds. Particularly preferred compounds (B) are primary and secondary amines.

When using different compounds (A) and/or (B), the different compounds (A) can be supplied in premixed form to an inlet opening of the reaction mixing pump, or via separate inlet openings. The same is true for the compounds (B).

The reaction of the compound(s) (A) with the compound(s) (B) can be carried out in a solvent system, but preferably without dilution by the processes known to the person skilled in the art. A reaction “without dilution” is understood herein as meaning one which takes place without or largely without the addition of solvents, with small amounts of solvents (less than 5% by weight, in particular less than 2% by weight, based on the weight of the reaction mixture) introduced into the reaction mixture for example via any catalyst used or to increase the reaction rate as a consequence of polarity increase—being ignored. If solvents are used, then these serve in particular to adapt the viscosity or to increase the reaction rate. The reaction temperature to be selected depends here also on the reactivity of the starting materials. Optionally, catalysts known to the person skilled in the art are used in order to increase the rate of the reaction.

Typical catalysts of the addition reactions according to the invention are basic or acidic catalysts, phosphines or lanthanoid compounds. Among the basic catalysts, particular preference is given to tertiary amines. Acidic catalysts which can be used are primarily Lewis acids such as boron trifluoride, zinc chloride, aluminum chloride or titanium tetrachloride. Lanthanoid compounds are described e.g. in DE 69607568.

The catalysts are advantageously dissolved or dispersed in the compound(s) (A) or (B) before entering the reaction mixing pump.

In the preferred case, in which amines are used as nucleophile, the addition of a catalyst can preferably also be dispensed with since the amino compounds and their reaction products are themselves catalytically active.

In one embodiment, compound(s) (A) and compound(s) (B) are supplied to the reaction mixing pump in a ratio such that the ratio of carbon-carbon multiple bonds from compound(s) (A) to nucleophilic groups from compound(s) (B) is essentially equimolar, i.e. is 1:1.1 to 1.1:1.

In another embodiment, it may be advantageous to supply compound(s) (A) and compound(s) (B) to the reaction mixing pump in a ratio such that the ratio of carbon-carbon multiple bonds from compound(s) (A) to nucleophilic functionalities form compound(s) (B) is selected such that the alkenically or alkynically unsaturated functionalities are present in an excess, in particular an excess of more than 10 mol %. The end product then still contains alkenically or alkynically unsaturated functions which
(a) are required for the ultimate use of the product, for example radically polymerizing or crosslinking systems, or
(b) further react with the species formed or
(c) are reacted in a downstream reaction mixing pump with one or more further compound(s) (A) which differ from the compound(s) (A) used in the first step. In this way, it is also possible to operate more than two reactors or reaction mixing pumps in series.

A preferred example of case (b) was the addition of primary amino groups onto activated carbon-carbon double bonds which are present as secondary amino groups after the addition. The secondary amino groups are likewise capable of an addition reaction onto an activated carbon-carbon double bond, meaning that multiple addition products can be produced here.

In another embodiment, it may be advantageous to introduce compound(s) (A) and compound(s) (B) into the reaction mixing pump in a ratio such that the ratio of carbon-carbon multiple bonds from compound(s) (A) to nucleophilic functionality from compound(s) (B) is selected such that nucleophilic functionalities are present in an excess, in particular an excess of more than 10 mol %; this may be advantageous in the case when polyfunctional compounds (A), e.g. polyamines, are present, of which only some of the functional groups are reacted. The end product then still contains nucleophilic donor groups which
(a) are required for the ultimate use of the product, for example in epoxide-containing or isocyanate-containing binder systems or in radically polymerizing or crosslinking (preferably acrylate-functional) systems, or
(b) are reacted in a downstream reaction mixing pump with one or more further compound(s) (B) which differ from the compound(s) (B) used in the first step. In this way, more than two reactors or reaction mixing pumps can also be operated in series.

In a preferred embodiment of the invention, the temperature of the reaction mixture in the reactor is 0-200° C., preferably 10-150° C., and particularly preferably 20-120° C.

The quotient of total volume of the reaction mixture present in the reactor and the total volume stream of the reaction mixture discharged from the reactor in the form of the product stream is considered to be a measure of the residence time. The relevant relatively short residence times ensure that, despite the relatively high temperatures, undesired secondary reactions are only evident to a slight extent.

The compound(s) (A) and the compound(s) (B) are in each case supplied to the reactor normally with an entry temperature of 0-200° C., preferably 10-100° C., and particularly preferably 20-50° C. The difference between the exit temperature (upon exiting from the reactor) of the reaction mixture and this entry temperature is in most cases 0 to 100° C., preferably 10 to 50° C. Typically, the heating capacity, based on the heat in the reactor introduced into the reaction mixture from outside, is 1 to 10000 watts per kg, preferably 80 to 2000, particularly preferably approximately 100 to 500 watts per kg.

The invention further provides a method for producing coated pulverulent or fibrous solids, where, in a first step, the method according to the invention for the continuous production of reaction products by addition reaction is carried out and, in a subsequent step, pulverulent or fibrous solids are coated with the reaction product of the method carried out in the first step. Typical pulverulent or fibrous solids are pigments and fillers, preferably inorganic fillers, and also glass fibers and carbon fibers. Nanoscale fillers (e.g. SiO₂, Al₂O₃, ZnO, carbon nanotubes) are a specific example of inorganic fillers. Effect pigments (e.g. based on metal pigments, for example made of aluminum, zinc or brass, and also pearlescent pigments) are a further specific example. The pigments or fillers in turn can be present before the coating in dry form or, for example, in the form of pigment pastes, cosmetic preparations, writing inks, printing inks or paints.

The invention also further provides a method for producing coatings or plastics, where, in a first step, the method according to the invention for the continuous production of reaction products by addition reaction is carried out and, in a subsequent step, the reaction product of the method carried out in the first step is incorporated into coating compositions or plastics. Coating compositions in the context of this invention are, for example, paints, adhesives, sealants, pouring compounds, but also in the widest sense writing inks and printing inks. Plastics can be for example thermoplastic or thermoset plastics.

The invention also further provides a method for producing coated material surfaces, where, in a first step, the method according to the invention for the continuous production of reaction products by addition reaction is carried out and, in a subsequent step, the reaction product of the method carried out in the first step is used for the coating and/or treatment of material surfaces. Material surfaces in the context of this invention are, for example, metallic surfaces, glass surfaces, plastic surfaces or ceramic surfaces.

The present invention also relates to the addition compounds which have been produced with the methods described above. These addition compounds include, in particular, also polymeric addition compounds, as described for example in Prog. Polym. Sci. 2006, 31, 487.

The present invention also discloses the use of the addition products produced by the method according to the invention described above as additives, for example as adhesion promoters and coupling agents, as wetting agents and dispersants, as surface-active, water-repellent or soil-repellent agents, such as, for example, antigraffiti agents, release agents or wetting agents. They can, for example, however also be used for the modification of polymers or resins or for the treatment of pigments or fillers.

Particularly when used as levelling and film-forming auxiliaries in automobile OEM paints or automobile repair paints, the addition compounds produced according to the invention play an important role. In such qualitatively high-value systems, the purest possible compounds should be used which also exhibit a minimum of intrinsic coloration. This is true in particular for non-pigmented clearcoats. Since the compounds obtained according to the invention make do using a minimal amount of catalyst, this prerequisite can be satisfied.

As well as the use as additive in aqueous and/or solvent-containing dispersions, in particular coating compositions such as paints, it is likewise possible to coat pulverulent or fibrous solids, such as pigments or fillers with the products obtained by the method according to the invention.

Consequently, the present invention finally also discloses pulverulent or fibrous solids which have been coated with the products obtained by the method according to the invention.

Such coatings of organic and inorganic solids are carried out in a known manner. For example, such methods are described in EP-A 0 270 126. Specifically in the case of pigments, coating of the pigment surface can take place during or after synthesis of the pigments, for example by adding the products obtained according to the invention to the pigment suspension. Pigments pretreated in this way exhibit ready incorporability into the binder system, an improved viscosity and flocculation behavior, as well as a good gloss compared with untreated pigments.

In general, the addition products according to the invention are suitable as interface-active compounds in paints, plastics, adhesives, pigment pastes, sealants, cosmetic preparations, ceramics, pouring compounds, printing inks or writing inks. The use as interface-active compound can take place, according to the application in question, for example as wetting agent and dispersant, adhesion promoter, coupling agent, emulsifier, release agent, antifoam, deaerator or as processing auxiliary.

The invention will be described in more detail below by reference to working examples.

### EXAMPLES

### Example 1 (According to the Invention)

### Reaction of a Polyethylene Glycol-200 Diacrylate (PEG200-DA) with Aminopropyltriethoxysilane (AMEO)

Firstly, a reaction mixing pump of the type HMR-40 (K-Engineering, Westoverledingen, Germany) and a thermostat of the type Huber K25-CC-NR for the post-reaction were brought to a working temperature of 80° C.

After the working temperatures were reached, the material streams were continuously conveyed from storage vessels 1 and 2 (1-AMEO): 5.9 g/min; PEG200-DA: 14.5 g/min) into the reaction chamber of the reaction mixing pump by means of pumps 1 and 2. The stabilizer used (2,6-di-tert-butyl-p-cresol) was dissolved beforehand in the PEG200-DA.

The reaction mixing pump was operated via a frequency converter with 30% of the maximum possible number of revolutions. During the reaction (continuously over 5 hours), a temperature of 78-83° C. was measured in the reaction space of the reaction mixing pump. For the post-reaction, the reaction mixture was fed via a suitable tube made of polytetrafluoroethylene through a heated bath of the thermostat.

The tube used for the post-reaction had an internal diameter of 6 mm and a length of 10 m. The total system volume (reaction mixing pump and downstream tube) was ca. 288 ml. The total reaction time (residence time in pump and tube) was ca. 15 min.

The conversion of amine determined by means of NMR was 100%.

### Example 2 (According to the Invention)

### Reaction of Dipropylene Glycol Diacrylate (DPGDA) and Trimethylolpropane Triacrylate (TMPTA) with Aminopropyltriethoxysilane (AMEO)

Firstly, a reaction mixing pump of the type HMR-40 (K-Engineering, Westoverledingen, Germany) and a thermostat of the type Huber K25-CC-NR for the post-reaction were brought to a working temperature of 40° C.

After the working temperatures were reached, the material streams were continuously conveyed from storage vessels 1 and 2 (DPGDA+TMPTA in the weight ratio 9-to-1:11.0 g/min; AMEO: 6.1 g/min) into the reaction chamber of the reaction mixing pump by means of pumps 1 and 2. The stabilizer used, 2,6-di-tert-butyl-p-cresol, was dissolved beforehand in the acrylate mixture (DPGDA+TMPTA). The reaction mixing pump was operated via a frequency converter with 30% of the maximum possible number of revolutions. During the reaction (continuously over 5 hours), a temperature of 38-43° C. was measured in the reaction space of the reaction mixing pump. For the post-reaction, the reaction mixture was fed via a suitable tube made of polytetrafluoroethylene through a heated bath of the thermostat.

The tube used for the post-reaction had an internal diameter of 6 mm and a length of 10 m. The total system volume was ca. 288 ml. The total reaction time was ca. 10 min.

The conversion of amine determined by means of NMR was 100%.

### Example 3 (According to the Invention)

### Reaction of a Propoxylated Neopentyl Glycol Diacrylate (2 mol of Propylene Oxide Per Neopentyl Glycol) with Triethylenetetramine (TETA) and 2-Ethylhexyl Acrylate (EHA), Weight Ratio 4:5:2

Firstly, a reaction mixing pump of the type HMR-40 (K-Engineering, Westoverledingen, Germany) and a thermostat of the type Huber K25-CC-NR for the post-reaction were brought to room temperature, 25° C.

After the working temperatures were reached, the material streams were continuously conveyed from storage vessels 1 and 2 (TETA: 1.5 g/min; diacrylate and EHA: 3.4 g/min) into the reaction chamber of the reaction mixing pump by means of pumps 1 and 2. The reaction mixing pump was operated via a frequency converter with 20-40% of the maximum possible number of revolutions. During the reaction (continuously over 5 hours), a temperature of 25-30° C. was measured in the reaction space of the reaction mixing pump. No post-reaction is performed.

This gives a product with the following analytical data: viscosity (plate/cone) 36 Pas (20° C.), 2000 mPas (60° C.); density 1.049 g/ml; refractive index 1.4851.

1. A method for the continuous production of reaction products by addition reaction, where at least one compound (B), which has at least one nucleophilic group, is added onto at least one compound (A), which has at least one activated alkenic or activated alkynic carbon-carbon multiple bond, where the activation of the carbon-carbon multiple bond takes place by means of an electron-withdrawing substituent which is adjacent to the carbon-carbon double bond or carbon-carbon triple bond, wherein the reaction takes place in a reaction mixing pump.

2. The method as claimed in claim 1, wherein the reaction mixing pump is of the peripheral wheel pump type and is equipped with:
(a) a rotationally symmetrical mixing chamber composed of a circumferential wall and two faces, which have annular channels fluidically joined to one another,
(b) at least one inlet opening to the mixing chamber, via which the compound(s) (A) are introduced,
(c) at least one inlet opening to the mixing chamber, via which compound(s) (B) are introduced,
(d) a magnet-coupling-driven mixing rotor in the mixing chamber, which has edge breaks symmetrically arranged on the face which form pressure cells with the annular channels on the faces of the mixing chamber, and where the pressure cells are joined together via connecting bores in the mixing rotor, and
(e) an outlet opening of the mixing chamber, via which the reaction mixture and/or the product are discharged from the reaction mixing pump, and
(f) a thermally regulatable circuit thermally regulatable by means of an external heating or cooling unit.

3. The method as claimed in claim 2, where the reaction mixing pump is provided further (g) with an inlet opening for rinse liquids.

4. The method as claimed in claim 1, wherein the compound (A) has one of the general formulae (Ia) and (Ib): 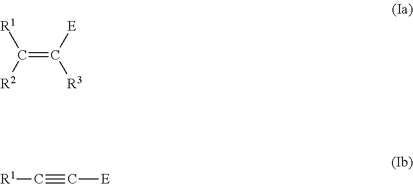
in which
E is an electron-withdrawing substituent, and R¹, R² and R³, independently of one another, are H, E, an aliphatic, aromatic or aliphatic-aromatic radical, and where R¹ and E can be joined together by ring closure.

5. The method as claimed in claim 4, where E is selected from the group consisting of the radicals COR, COOR, CONHR, CONR₂, CN, PO(OR)₂, pyridyl, SOR, SOOR, F or NO₂, where the radicals R, independently of one another, are H or an aliphatic, aromatic or aliphatic-aromatic radical.

6. The method as claimed in claim 5, where at least two of the radicals R¹, R² and R³ in the compound of the general formula (Ia) are hydrogen.

7. The method as claimed in claim 4 wherein:
(i) the radicals R¹, R² and R³ in the general formula (Ia) are hydrogen, or
(ii) the radicals R¹ and R² in the general formula (Ia) are hydrogen, R³ is a substituted or unsubstituted methyl radical, and E is a radical COOR, CONHR, CONR₂ or CN, or
(iii) two of the three radicals R¹, R² and R³ in the general formula (Ia) are hydrogen, and the third radical that is not hydrogen is a further group E, and the radicals R¹ and E are optionally bonded by ring closure formation.

8. The method as claimed in claim 1, where the compound (A) is selected from the group consisting of (meth)acrylates, (meth)acrylamides, acrylonitriles, maleic anhydride, maleic acid, its esters, amides and imides, itaconic acid, its esters and amides, cyanoacrylates, vinylsulfones, vinylphosphonates, vinyl ketones, nitroethylenes, α,β-unsaturated aldehydes, vinylpyridines, β-ketoacetylenes and acetylene esters.

9. The method as claimed in claim 4, where the compounds (A) carry two or more radicals CR¹R²═CR³COO.

10. The method as claimed in claim 1, where the compounds (A) are selected from the group consisting of hexanediol diacrylate, dipropylene glycol diacrylate, tripropylene glycol diacrylate, trimethylolpropane triacrylate, pentaerythritol triacrylate, pentaerythritol tetraacrylate, dimethylolpropane tetraacrylate, polyether acrylates, polyester acrylates, epoxy acrylates or urethane acrylates.

11. The method as claimed in claim 1, where the compound (B) has the general formula (II):
R⁴-D-H (II)
in which R⁴ is an aliphatic, aromatic or aliphatic-aromatic radical and D is CR′E, NR′, PR′ or S, where R′ is hydrogen or an aliphatic, aromatic or aliphatic-aromatic radical and E is an electron-withdrawing substituent.

12. The method as claimed in claim 1, where the compound (B) is selected from the group consisting of primary amines, secondary amines, thiols, phosphines and carbanion-forming compounds.

13. The method as claimed in claim 12, where the primary amines and secondary amines are polyamines or amines carrying further reactive radicals, where the reactive radicals are selected from the group consisting of hydrolyzable silyl groups or hydroxyl groups.

14. The method as claimed in claim 1, where the addition reaction is catalyzed with basic or acidic catalysts, phosphines or lanthanoid compounds.

15. A method for producing coated pulverulent or fibrous solids, where, in a first step, the method as claimed in claim 1 is carried out and, in a subsequent step, pulverulent or fibrous solids are coated with the reaction product of the method carried out in the first step.

16. A method for producing coatings or plastics, where, in a first step, the method as claimed in claim 1 is carried out, and, in a subsequent step, the reaction product of the method carried out in the first step is incorporated into coating compositions or plastics.

17. A method for producing coated material surfaces, where, in a first step, the method as claimed in claim 1 is carried out, and, in a subsequent step, the material surface is coated with the reaction product of the method carried out in the first step.

18. A method for the continuous production of reaction products by addition reaction comprising:
obtaining or providing at least one compound (B) comprising at least one nucleophilic group; and
contacting the at least one compound (B) with at least one compound (A) in a reaction mixing pump, wherein the at least one compound (A) comprises at least one activated alkenic or activated alkynic carbon-carbon multiple bond, where the activation of the carbon-carbon multiple bond takes place by means of an electron-withdrawing substituent which is adjacent to the carbon-carbon double bond or carbon-carbon triple bond;
wherein addition reaction products are obtained.
